(19) United States
(12) Patent Application Publication
(10) Pub. No.: US 2022/0268766 A1
(43) Pub. Date: Aug. 25, 2022

(54) HORMONE MASS SPECTROMETRIC DETECTION METHOD BASED ON ANTIBODY-COUPLED MAGNETIC BEADS ENRICHMENT TECHNOLOGY

(71) Applicants: Hangzhou Baichen medical Laboratory Co., Ltd, Hangzhou (CN); Hangzhou Baichen Medical Instrument Co., Ltd, Hangzhou (CN)

(72) Inventors: Qiang Gao, Hangzhou (CN); Chaochao Wu, Hangzhou (CN); Jianyou Xue, Hangzhou (CN)

(21) Appl. No.: 17/454,806

(22) Filed: Nov. 12, 2021

(30) Foreign Application Priority Data
Feb. 25, 2021 (CN) 202110209221 .X

(51) Int. Cl. G01N 33/543; G01N 33/74; G01N 30/72

(52) U.S. Cl. CPC G01N 33/54326; G01N 33/743; G01N 30/72; G01N 2030/027

(57) ABSTRACT

A hormone mass spectrometric detection method based on antibody-coupled enrichment technology. The method includes the following steps: coupling antibodies and magnetic beads, adding a high-concentration methanol solution, removing endogenous hormone small molecule compounds carried on the antibodies by vortex, then restoring the activity of the antibodies to obtain antibody magnetic beads that can enrich hormone small molecule compounds. The antibodies are immobilized by magnetic beads and cooperate with the 80% methanol aqueous solution to elute hormone small molecule compounds, and then the hormone small molecule compounds are tested by mass spectrometry, thereby improving the specificity of test.

### CROSS-REFERENCE TO RELATED APPLICATIONS

This is a U.S. patent application which claims the priority and benefit of Chinese patent application number 202110209221 .X, filed on Feb. 25, 2021, the disclosure of which is incorporated herein by reference in its entirety.

### FIELD OF THE INVENTION

The present invention relates to the field of hormone test, in particular to a hormone mass spectrometric detection method based on antibody-coupled magnetic bead enrichment technology.

### BACKGROUND OF THE INVENTION

At present, endogenous hormones are usually tested in clinic by an antibody-based chemiluminescence method, in which substances to be tested are captured using a specific antibody, and the substances to be tested are separated through antibody magnetic beads and then tested by an enzyme-labeled method. However, this method has the defects of poor specificity and narrow linear range, and cannot accurately test concentrations of target compounds such as hormones. Liquid chromatography-mass spectrometry (LC-MS/MS) detection as a new type of detection technology has high specificity and wide linear range, can test multiple indicators at the same time, and can specifically overcome the defects of the chemiluminescence method when testing hormones.

However, samples for testing hormones in present LC-MS/MS are pretreated by a non-specific enrichment method, so the samples contain more unnecessary matrix substances, which will cause a relatively high matrix effect during test. In addition, it is difficult to separate the substances with similar structures. Therefore, the LC-MS/MS is not widely used in hormone test.

In addition, the present methods are difficult to apply to test hormone endogenous small molecule compounds expressed by antibodies. When antibodies are expressed in serum, hormone endogenous small molecule compounds will be produced, and the antibodies will bind tightly to the hormone endogenous small molecule compounds. The hormone endogenous small molecule compounds cannot be effectively separated by simple antibody purification steps, so that such hormones cannot be accurately tested subsequently.

### SUMMARY OF THE INVENTION

The objective of the present invention is to provide a hormone mass spectrometric detection method based on antibody-coupled enrichment technology, in which antibodies are immobilized by magnetic beads, 10% methanol is used as an enrichment buffer system, hormone small molecule compounds enriched on the antibody magnetic beads are eluted with an 80% methanol aqueous solution, and then the hormone small molecule compounds are tested by mass spectrometry, thereby improving the specificity of test.

According to a first aspect of the present invention, a hormone mass spectrometric detection method based on antibody-coupled enrichment technology is provided, including the following steps:

preparing antibody magnetic beads: coupling antibodies and magnetic beads, standing the same on a magnetic stand, discarding the supernatant, adding a 50%-80% methanol solution and removing endogenous hormones carried on the antibodies by vortex, discarding the supernatant, then restoring the activity of the antibodies to obtain antibody magnetic beads that can enrich hormone small molecule compounds, and adding test serum or a standard curve sample to obtain a first solution;

enriching test samples: adding an internal standard to the first solution and incubating the same at room temperature by vortex, and adding a 5-20% methanol solution for washing, to obtain antibody magnetic beads enriched with hormone small molecule compounds;

eluting the test samples: placing the antibody magnetic beads enriched with hormone small molecule compounds in an 80% methanol aqueous solution and re-suspending the same by vortex to obtain an eluent containing the hormone small molecule compounds; and

testing the hormone small molecule compounds by mass spectrometry: placing the eluent under the set mass spectrometry conditions for test.

In some embodiments, the activity of antibodies can be restored after antibodies and magnetic beads are coupled and endogenous hormone small molecule compounds carried on the antibodies are removed, including a step of: placing the antibody magnetic beads in organic solvent aqueous solutions with concentrations from high to low for gradient washing. Specifically, the antibody magnetic beads can be first washed 3 to 5 times with the 80% methanol aqueous solution, and then sequentially washed once with a 65%-55% organic solvent aqueous solution, a 55-45% organic solvent aqueous solution, a 45-35% organic solvent aqueous solution, a 35-25% organic solvent aqueous solution, a 25-15% organic solvent aqueous solution and a 15-5% organic solvent aqueous solution, and finally the antibody magnetic beads are stored in the 15-5% organic solvent aqueous solution. When the activity of antibodies is restored by organic solvent aqueous solutions with different concentrations in the antibody activity restoration stage, the antibody magnetic beads are re-suspended in the corresponding concentration of organic solvent aqueous solution and mixed upside down for 2-4 minutes, and the current concentration of organic solvent aqueous solution is discarded.

The organic solvent aqueous solution is a methanol aqueous solution.

Enriching test samples: adding test serum or a standard curve sample prepared with 10% methanol, and an internal standard prepared with 10% methanol to the prepared antibody magnetic beads, incubating the same at room temperature by vortex for 30 minutes, standing the same on a magnetic stand and discarding the supernatant, adding a 10% methanol solution and washing upside down for 1-2 minutes, discarding the supernatant, and repeating the washing for 3 times, to obtain antibody magnetic beads enriched with hormone small molecule compounds.

In the stage of eluting the test samples, the antibody magnetic beads enriched with hormone small molecule compounds are placed in an 80% methanol aqueous solution and re-suspended by vortex for 1-2 minutes to obtain an eluent containing the hormone small molecule compounds. This solution uses 10% methanol as the enrichment buffer system instead of PBS, and uses magnetic beads combined with an 80% methanol aqueous solution to elute hormone small hormone compounds on the antibodies. In the prior art, the magnetic beads are mostly used to purify antibodies, but in this solution, the magnetic beads do not purify antibodies, but help to immobilize the antibodies and cooperate with the 80% methanol aqueous solution to elute the hormone small molecule compounds adsorbed on the antibodies.

Specifically, antibodies bind to small molecule antigens such as endogenous hormones during expression. Both the endogenous hormone small molecule antigens and the antibodies are components of soluble organic solvents. In this solution, the magnetic beads immobilize the antibodies but not the endogenous hormone small molecule antigens, and the endogenous small molecule antigens are eluted from the antibodies with the 80% methanol aqueous solution. In this solution, after the endogenous small molecule antigens carried on the antibodies are eluted with the 80% methanol aqueous solution, the activity of antibodies can still be maintained. In most of the present reports, antigens are eluted with milder reagents (such as 0.1M glycine, glycine eluent with pH 3.0, citric acid eluent, etc.), but the elution effect is poor. There are two antigen binding sites on an antibody, one molecule of antibody can bind two molecules of antigen, the antibody is a protein, a high-concentration organic reagent can denature the antibody, and the antigen binding sites are destroyed after the antibody is denatured, so that the antigen and the antibody are eluted. In addition, the high-concentration organic reagent can remove the hydrophobic effect. The applicant has done a lot of verifiable experiments to verify that the 80% methanol aqueous solution can not only elute the antigen on the antibody, but the eluted antibody can restore its activity under the action of the gradient methanol aqueous solutions.

In the stage of enriching test samples, a PBS mixed solution or a 10% methanol aqueous solution may be used. Preferably, the 10% methanol aqueous solution enriches hormone small molecule compounds. Steroid hormones have poor solubility in PBS, but can be fully dissolved in 10% methanol; and the PBS is nearly neutral, and the 10% methanol is also nearly neutral.

It is worth mentioning that a variety of antibody-coupled magnetic beads may be mixed in the stage of enriching test samples without affecting the activity of antibodies, that is, at least two kinds of antibody-coupled magnetic beads are mixed to improve the enrichment effect.

In the mass spectrometric detection stage, the eluent is dried and reconstituted or directly transferred to a microplate and tested by a Waters TQD mass spectrometer, and different mass spectrometric conditions are provided for different hormone small molecule compounds.

The hormone mass spectrometric detection method based on antibody-coupled enrichment technology in this solution is particularly suitable for testing steroid hormones and other small molecule compounds.

In an embodiment of this solution, four antibodies: cortisol, testosterone, progesterone, and aldosterone, are used. Hormone small molecule compounds such as aldosterone, cortin, cortisol, 11-deoxycortisol, 11-deoxycorticosterone, androstenedione, testosterone, and progesterone are correspondingly tested.

In the second aspect, a hormone mass spectrometric detection kit based on antibody-coupled enrichment technology is provided, including: magnetic beads, antibodies, a 50-80% methanol solution, a 5-20% methanol solution for washing, organic solvent aqueous solutions with concentrations from high to low, and an 80% methanol aqueous solution, and using the hormone mass spectrometric detection method based on antibody-coupled enrichment technology according to any one of claims 1 to 9 for hormone mass spectrometric detection.

Compared with the prior art, this technical solution has the advantages that hormone small molecule compounds are enriched by antibody magnetic beads and eluted with an 80% methanol aqueous solution, and the eluted hormone small molecule compounds can be tested by mass spectrometry.

### BRIEF DESCRIPTION OF THE DRAWINGS

FIG. 1 is mass spectra of the hormone small molecule compounds including aldosterone, cortisol, 11-deoxycortisol, 11-deoxycorticosterone, androstenedione, testosterone, and progesterone.

FIG. 2 is mass spectra of the hormone small molecule compounds including aldosterone, cortisol, 11-deoxycortisol, 11-deoxycorticosterone, androstenedione, testosterone, and progesterone.

FIG. 3 is mass spectra of the hormone small molecule compounds including aldosterone, cortisol, 11-deoxycortisol, 11-deoxycorticosterone, androstenedione, testosterone, and progesterone.

FIG. 4 is mass spectra of the hormone small molecule compounds including aldosterone, cortisol, 11-deoxycortisol, 11-deoxycorticosterone, androstenedione, testosterone, and progesterone.

FIG. 5 is mass spectra of the hormone small molecule compounds including aldosterone, cortisol, 11-deoxycortisol, 11-deoxycorticosterone, androstenedione, testosterone, and progesterone.

FIG. 6 is mass spectra of the hormone small molecule compounds including aldosterone, cortisol, 11-deoxycortisol, 11-deoxycorticosterone, androstenedione, testosterone, and progesterone.

FIG. 7 is mass spectra of the hormone small molecule compounds including aldosterone, cortisol, 11-deoxycortisol, 11-deoxycorticosterone, androstenedione, testosterone, and progesterone.

FIG. 8 is linear graphs of the hormone small molecule compounds including aldosterone, cortisol, 11-deoxycortisol, 11-deoxycortisol, androstenedione, testosterone, and progesterone.

FIG. 9 is linear graphs of the hormone small molecule compounds including aldosterone, cortisol, 11-deoxycortisol, 11-deoxycortisol, androstenedione, testosterone, and progesterone.

FIG. 10 is linear graphs of the hormone small molecule compounds including aldosterone, cortisol, 11-deoxycortisol, 11-deoxycortisol, androstenedione, testosterone, and progesterone.

FIG. 11 is linear graphs of the hormone small molecule compounds including aldosterone, cortisol, 11-deoxycortisol, 11-deoxycortisol, androstenedione, testosterone, and progesterone.

FIG. 12 is linear graphs of the hormone small molecule compounds including aldosterone, cortisol, 11-deoxycortisol, 11-deoxycortisol, androstenedione, testosterone, and progesterone.

FIG. 13 is linear graphs of the hormone small molecule compounds including aldosterone, cortisol, 11-deoxycortisol, 11-deoxycortisol, androstenedione, testosterone, and progesterone.

FIG. 14 is linear graphs of the hormone small molecule compounds including aldosterone, cortisol, 11-deoxycortisol, 11-deoxycortisol, androstenedione, testosterone, and progesterone.

### DETAILED DESCRIPTION OF THE EMBODIMENTS

A clear and complete description will be made to the technical solutions in the embodiments of the present invention below with reference to the accompanying drawings in the embodiments of the present invention. Apparently, the embodiments described are only part of the embodiments of the present invention, not all of them. Based on the embodiments of the present invention, all other embodiments obtained by those of ordinary skill in the art shall fall within the protection scope of the present invention.

It can be understood that the term “a” should be understood as “at least one” or “one or more”, that is, in one embodiment, the number of an element may be one, and in another embodiment, the number of the element may be more than one, so the term “one” cannot be understood as a restriction on the number.

The following illustrates the test effects of a hormone mass spectrometric detection method based on antibody-coupled magnetic bead enrichment technology provided by this solution in conjunction with examples.

### EXAMPLE 1

### Antibodies Contained Endogenous Hormone Small Molecule Compounds

### Preparation of Reagents:

Four antibodies used: cortisol, testosterone, progesterone and aldosterone, purchased from Thermo; methanol, purchased from Merck, and mixed with water to prepare an 80% methanol aqueous solution;

Preparation of experimental instruments:

Self-made magnetic stand for 1.5 ml centrifuge tubes; 1.5 ml centrifuge tubes; vortex oscillator (Scientific Industries*Vortex-Genie2 Company, Vortex-Genie 2); Waters TQS mass spectrometer (Waters Company);

### Test Process:

5 μl of antibody (aldosterone, progesterone, testosterone or cortisol) was taken into a 1.5 ml centrifuge tube, 100 μl of 80% methanol aqueous solution was added, the centrifuge tube was placed in the vortex oscillator and whirled for 3 minutes, 12000 g was centrifuged at 4° C. for 10 minutes, 50 μl of the supernatant was transferred to a 96-well plate, 50 μl of water was added, and test was performed on Waters TQS after mixing.

### Mass Spectrometry and Chromatography Detection Methods

### 1. Chromatographic Conditions

1. Models and specifications of chromatographic column and pre-column: Waters HSS T3 (2.1*50 mm, 1.8 μm), HSS T3 (2.1*5.0 mm, 1.8 μm).

2. Mobile phase A: 0.1% formic acid, 2 mM ammonium acetate aqueous solution; mobile phase B: 0.1% formic acid, 2 mM ammonium acetate methanol solution.

2. Gradient elution program: 0 minute: A: B=55: 45; 4.0 minutes: A: B=40: 60; 6.5 minutes: A: B=25: 75; 7.5 minutes: A: B=10: 90; 7.6 minutes: A: B=55: 45; 8.0 minutes: A: B=55: 45.

3. Flow rate: 0.45 mL/min; column temperature: 45° C.; injection: 10 μL.

### 2. Mass Spectrometry Conditions

1. Model and scan mode of mass spectrometer: I-class Waters TQS, positive ion mode

2. Capillary voltage: 3.2 kv; desolvation temperature: 650° C.; desolvation gas: 800 L/hr; cone gas: 0 L/hr;

3. Scan mode: multiple reaction monitoring (MRM), mass spectrometry parameters of each compound were as follows:

| | | | Cone | Collision |
|---|---|---|---|---|
| Compound name | Parent ion | Daughter ion | voltage | voltage |
| Aldosterone | 361.17 | 343.5 | 40 | 20 |
| Aldosterone-d7 | 369.3 | 351.4 | 40 | 20 |
| Cortisone | 361.2 | 163.1 | 40 | 36 |
| Cortisone-d7 | 369.3 | 169.2 | 40 | 22 |
| Cortisol | 363.2 | 121 | 30 | 36 |
| Cortisol-d4 | 367.2 | 121.05 | 40 | 20 |
| 11-deoxycortisol | 347.2 | 109.3 | 30 | 40 |
| 11-deoxycortisol-d5 | 352.3 | 100.4 | 45 | 25 |
| Androstenedione | 287.26 | 97.08 | 24 | 30 |
| Androstenedione-C3 | 290.26 | 100.1 | 24 | 20 |
| Testosterone | 289.33 | 109.09 | 40 | 15 |
| Testosterone-d3 | 292.2 | 109.1 | 40 | 20 |
| 11-deoxycorticosterone | 331.2 | 109.17 | 30 | 30 |
| 11-deoxycorticosterone-d7 | 338.2 | 100.1 | 40 | 32 |
| Progesterone | 315.20 | 109.10 | 45 | 70 |
| Progesterone-d9 | 324.20 | 100.10 | 40 | 40 |

### Test Results:

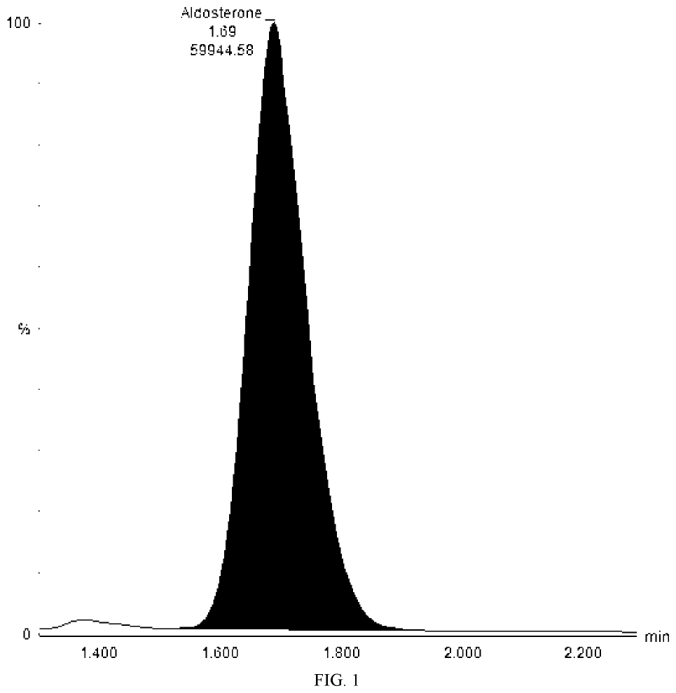

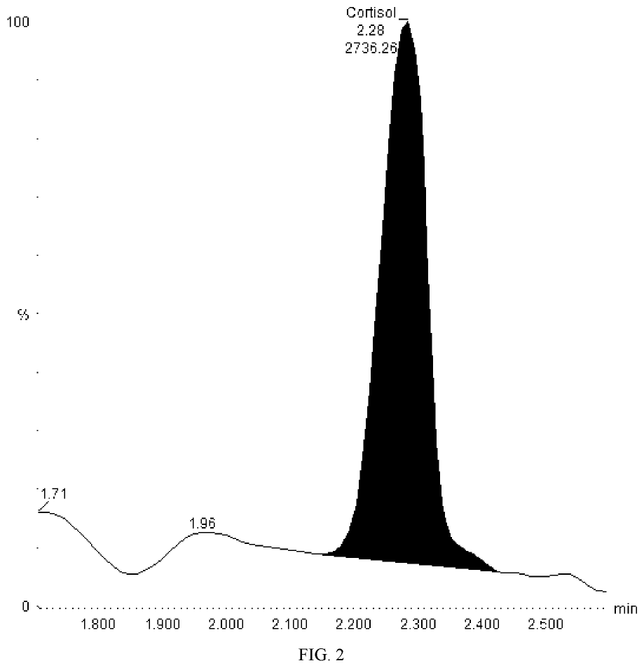

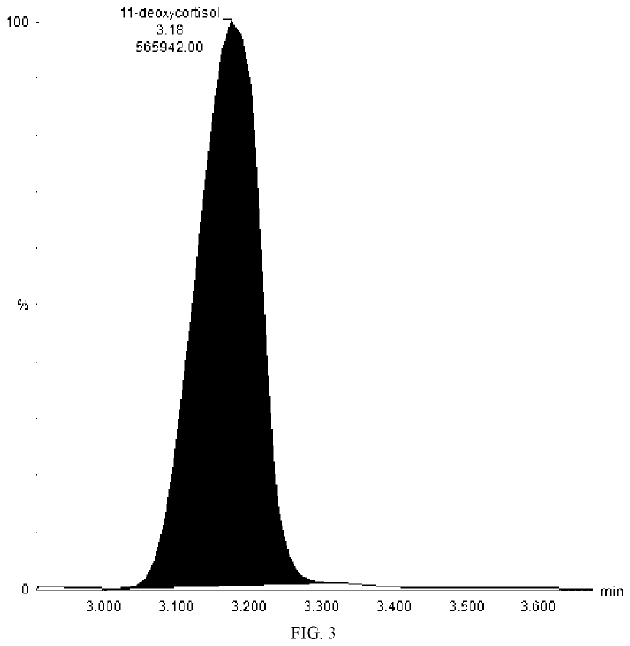

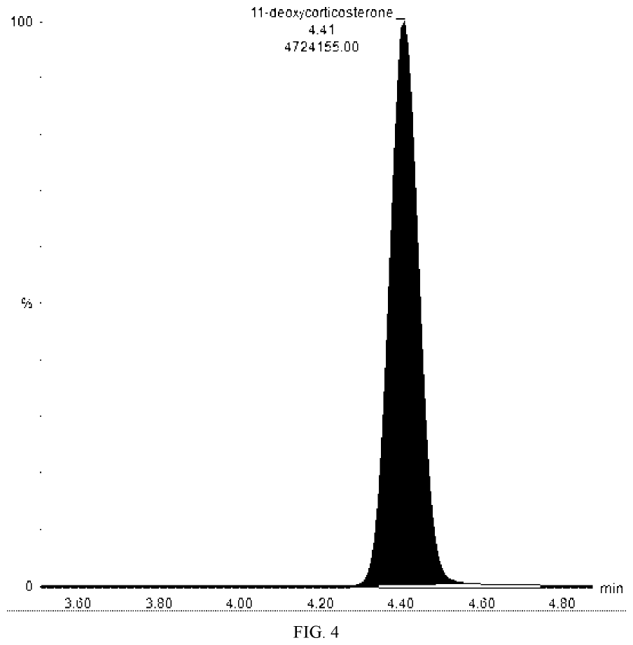

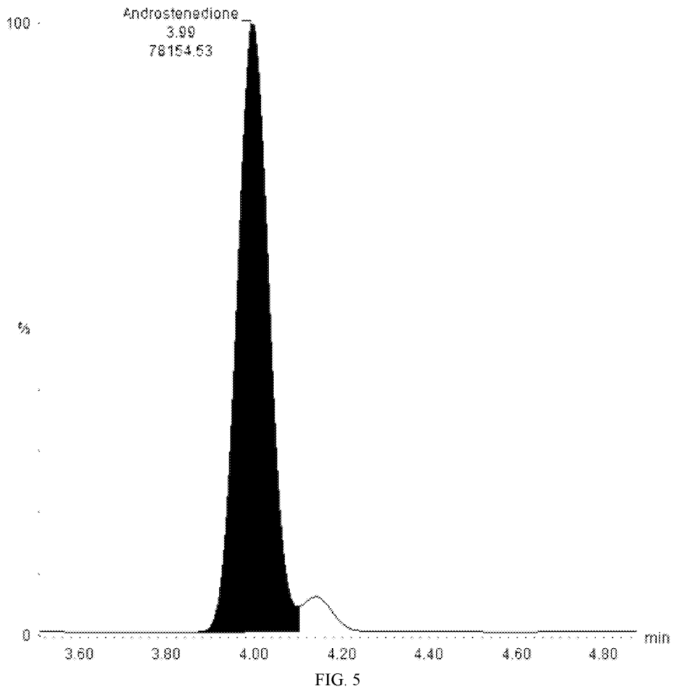

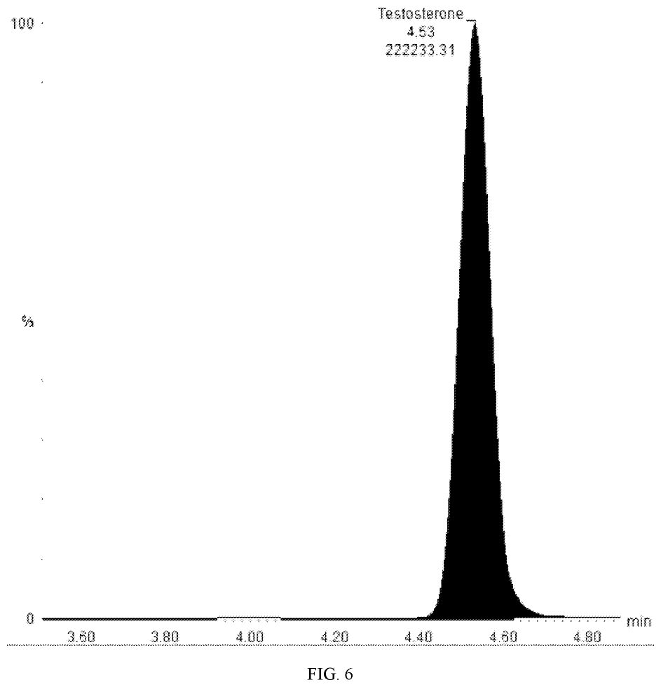

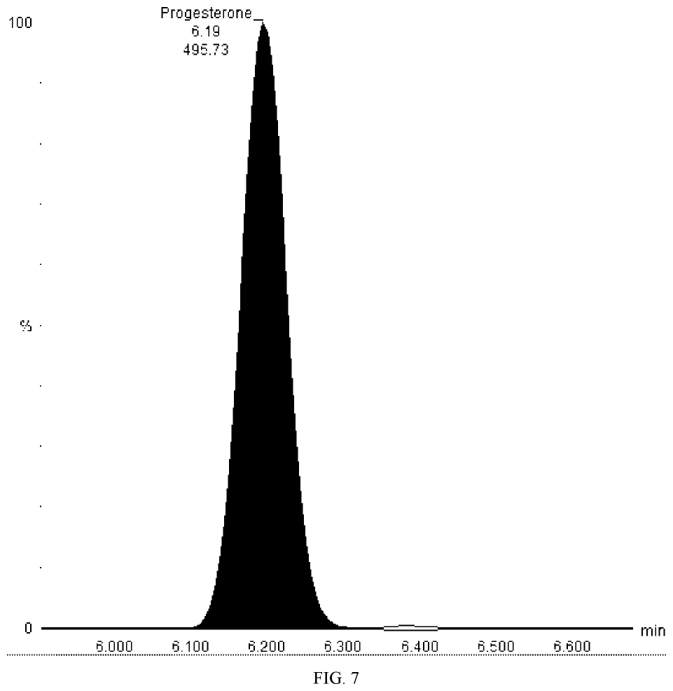

All four antibodies can test endogenous hormone small molecule compounds, including aldosterone, cortisol, 11-deoxycortisol, 11-deoxycorticosterone, androstenedione, testosterone, and progesterone. The mass spectra of the hormone small molecule compounds tested were shown in FIGS. 1 to 7. The peak area data of the hormone small molecule compounds were shown in Table 1:

TABLE 1
Peak area of endogenous hormones on antibodies

| Type of | Test index | | | | | | |
|---|---|---|---|---|---|---|---|
| Antibody | Aldosterone | Cortisol | 11-deoxycortisol | Androstenedione | 11-deoxycorticosterone | Testosterone | Progesterone |
| Aldosterone | 59944.58 | 412.63 | 8267.17 | 397.21 | 10454.77 | 0.00 | 0.00 |
| antibody | | | | | | | |
| Cortisol | 10318.14 | 2736.26 | 565942.00 | 612.74 | 74755.09 | 0.00 | 0.00 |
| antibody | | | | | | | |
| Testosterone | 0.00 | 0.00 | 486.70 | 78154.53 | 7795.91 | 222233.31 | 0.00 |
| antibody | | | | | | | |
| Progesterone | 124763.56 | 0.00 | 301585.25 | 3956.73 | 4724155.00 | 550.20 | 495.73 |
| antibody | | | | | | | |

### EXAMPLE 2

### Both 0.1M glycine pH2.8 eluent and 20% methanol Solution Cannot Effectively Remove Endogenous Hormones on Antibody-Coupled Magnetic Beads

### Preparation of Reagents:

Glycine was purchased from Sigma and prepared into 0.1M glycine with pH 2.8; phosphate buffer solution (PBS) was purchased from Sigma, and deionized water was purchased from Merck; carbodiimide was purchased from Sigma, and added with water to prepare 10 mg/mL; Tris was purchased from Sigma and added with water to prepare 50 mM Tris-HCl with pH 7.4, and carboxyl magnetic beads were purchased from Enriching Biotechnology LTD. Other reagents were the same as those in Example 1.

Preparation of Experimental Instruments: same as Example 1

### Test Process:

1. Antibody and magnetic bead coupling: magnetic beads were mixed uniformly, 10 mg of magnetic beads were added to a 1.5 mL EP tube, the supernatant was removed by magnetic separation on a magnetic stand, deionized water was added and uniform mixing was performed, the supernatant was removed by magnetic separation, and this process was repeated 2 times. 100 μL of PBS was added to re-suspend the magnetic beads, the supernatant was removed by magnetic separation, and this process was repeated 2 times. 50 μL of PBS was added to re-suspend the magnetic beads, 1 mg of (aldosterone, progesterone, testosterone or cortisol) antibodies were added respectively, and the both were mixed at room temperature for 30 minutes while whirling. 10 μL of 10 mg/mL carbodiimide solution currently prepared was added, and PBS was supplemented to make a total volume 1000 μL of the system. The coupling was performed by vortex at room temperature for 1 hour, and the supernatant was removed by magnetic separation. 100 μL of quenching buffer (50 mM Tris-HCl, pH 7.4) was added to re-suspend the magnetic beads, vortex mixing was performed for 30 minutes, and the supernatant was removed by magnetic separation; 1000 μL of PBS was added, the magnetic beads were mixed and re-suspended, the supernatant was removed by magnetic separation, this process was repeated 2 times, and the magnetic beads were dispersed in 1000 μL of 10% methanol and stored at 4° C. to obtain antibody-coupled magnetic beads.

2. 30 μl of 1 mg/ml antibody-coupled magnetic beads (aldosterone, progesterone, testosterone or cortisol) was added with 100 μl of 0.1M Gly with pH 2.8 or 20% methanol for re-suspending, the endogenous hormones on the antibodies were eluted upside down several times, the antibody-coupled magnetic beads were placed on the magnetic stand and magnetically separated, 100 μl of the supernatant was transferred to a 96-well plate, the elution was repeated 2 times, finally elution was performed once again with 80% methanol, 50 μl of the supernatant in each step was transferred to the 96-well plate, and 50 μl of water was added, followed by uniform mixing and Waters TQS mass spectrometric detection.

### Test Results:

The endogenous small molecule compounds cannot be well eluted in case of washing 3 times (Glyl, 2, 3) with 0.1M glycine pH 2.8 eluent. The peak area data of the hormone small molecule compounds tested were shown in Table 2.

The endogenous small molecule compounds also cannot be well eluted with 20% methanol. The peak area data of the hormone small molecule compounds tested were shown in Table 3:

TABLE 2
Elution peak area of endogenous hormones on antibodies with 0.1M glycine pH 2.8 eluent

| Type of | Elution | Test index | | | | | | |
|---|---|---|---|---|---|---|---|---|
| antibody | condition | Aldosterone | Cortisol | 11-deoxycortisol | Androstenedione | 11-deoxycorticosterone | Testosterone | Progesterone |
| Aldosterone | 80% methanol | 52631.48 | 0.00 | 0.00 | 212.31 | 4892.52 | 0.00 | 0.00 |
| antibody | Gly-3 | 500.42 | 0.00 | 0.00 | 0.00 | 273.44 | 0.00 | 0.00 |
| | Gly-2 | 539.47 | 0.00 | 0.00 | 0.00 | 341.73 | 0.00 | 0.00 |
| | Gly-1 | 497.49 | 0.00 | 0.00 | 0.00 | 341.64 | 0.00 | 0.00 |
| Cortisol | 80% methanol | 565.24 | 5003.12 | 1013.60 | 514.97 | 69988.34 | 0.00 | 0.00 |
| antibody | Gly-3 | 0.00 | 0.00 | 0.00 | 0.00 | 1549.44 | 0.00 | 0.00 |
| | Gly-2 | 0.00 | 0.00 | 0.00 | 0.00 | 1876.13 | 0.00 | 0.00 |
| | Gly-1 | 0.00 | 0.00 | 0.00 | 61.89 | 2222.88 | 0.00 | 0.00 |
| Testosterone | 80% methanol | 0.00 | 0.00 | 0.00 | 2183.13 | 578.31 | 109522.75 | 0.00 |
| antibody | Gly-3 | 0.00 | 0.00 | 0.00 | 2702.03 | 0.00 | 424.91 | 0.00 |
| | Gly-2 | 0.00 | 0.00 | 0.00 | 2900.78 | 0.00 | 302.90 | 0.00 |
| | Gly-1 | 0.00 | 0.00 | 0.00 | 6112.63 | 0.00 | 429.79 | 0.00 |
| Progesterone | 80% methanol | 5366.78 | 0.00 | 0.00 | 2001.95 | 3373284.50 | 0.00 | 501.60 |
| antibody | Gly-3 | 3918.43 | 0.00 | 0.00 | 261.61 | 148261.30 | 0.00 | 0.00 |
| | Gly-2 | 11231.23 | 0.00 | 0.00 | 601.95 | 162603.48 | 0.00 | 0.00 |
| | Gly-1 | 53394.37 | 0.00 | 0.00 | 910.39 | 191064.14 | 0.00 | 0.00 |

TABLE 3
Elution peak area of endogenous hormones on antibodies with 20% methanol eluent

| Type of | Elution | Test index | | | | |
|---|---|---|---|---|---|---|
| antibody | condition | Aldosterone | Cortisol | 11-deoxycortisol | Androstenedione | 11-deoxycorticosterone |
| Aldosterone | 80% methanol | 4077.381 | 0.00 | 0.00 | 212.31 | 24464.31 |
| antibody | 20% methanol | 591.092 | 0.00 | 0.00 | 0.00 | 4306.02 |
| Cortisol | 80% methanol | 0.00 | 3614.79 | 59769.55 | 514.97 | 0.00 |
| antibody | 20% methanol | 0.00 | 0.00 | 42104.86 | 0.00 | 206.40 |

### EXAMPLE 3

80% methanol can effectively remove endogenous hormones on antibody-coupled magnetic beads without affecting the activity of antibodies. 10% methanol was used as an enrichment system and used with 4 kinds of antibodies to achieve better enrichment effect.

### Preparation of Reagents:

All hormone compounds and internal standards were purchased from Sigma, other reagents were the same as those in Example 1 or 2, and 10% methanol was used to prepare internal standards (Table 4) and calibrators (Table 5).

TABLE 4
Internal standard concentration of each compound

| | Cortin- | Cortisol- | 11-deoxycortisol- | Androstenedione- | Testosterone- | 11-deoxycorticosterone- | Aldosterone- |
|---|---|---|---|---|---|---|---|
| Name | d7 | d4 | d5 | 13C3 | 13C3 | d7 | d7 |
| Concentration (ng/mL) | 4.0 | 4.0 | 2.0 | 2.0 | 2.0 | 0.2 | 0.2 |

TABLE 5
Concentration of each calibrator compound

| | Concentration of each calibrator (ng/mL) | | | | | | | |
|---|---|---|---|---|---|---|---|---|
| Name | Line 8 | Line 7 | Line 6 | Line 5 | Line 4 | Line 3 | Line 2 | Line 1 |
| Cortin | 500 | 100 | 50 | 10 | 5 | 1 | 0.5 | 0.25 |
| Cortisol | 500 | 100 | 50 | 10 | 5 | 1 | 0.5 | 0.25 |
| 11-deoxycortisol | 50 | 10 | 5 | 1 | 0.5 | 0.1 | 0.05 | 0.025 |
| Androstenedione | 20 | 4 | 2 | 0.4 | 0.2 | 0.04 | 0.02 | 0.01 |
| Testosterone | 50 | 10 | 5 | 1 | 0.5 | 0.1 | 0.05 | 0.025 |
| 11-deoxycorticosterone | 20 | 4 | 2 | 0.4 | 0.2 | 0.04 | 0.02 | 0.01 |
| Aldosterone | 20 | 4 | 2 | 0.4 | 0.2 | 0.04 | 0.02 | 0.01 |

Preparations of other reagents and experimental instruments were the same as those in Example 1 or 2.

### Test Process:

Antibody-coupled magnetic beads were prepared in the same way as described in Example 2.

1. Removal of endogenous hormones on antibody-coupled magnetic beads: 1 ml of 1 mg/ml antibody-coupled magnetic beads (aldosterone, progesterone, testosterone or cortisol) was respectively placed on a magnetic stand and magnetically separated, the supernatant was discarded, 1 ml of 80% methanol was added for re-suspending, the endogenous hormones on the antibodies were removed by upside down several times, the supernatant was discarded after magnetic separation, the antibody-coupled magnetic beads were washed twice with 80% methanol, the supernatant was discarded after magnetic separation, then the antibody-coupled magnetic beads were washed once in a gradient manner with 1 ml of 70%, 60%, 50%, 30%, 20% and 10% methanol respectively to restore the activity of the antibody-coupled magnetic beads, and finally the antibody-coupled magnetic beads were stored in the 10% methanol.

2. Experiment of mixing four kinds of antibodies: 20 μl of 1 mg/ml antibody-coupled magnetic beads (aldosterone, progesterone, testosterone, cortisol) was respectively taken or 20 μl of each of four kinds of 1 mg/ml antibody-coupled magnetic beads was taken and mixed, 200 μl of standard 6 (Table 5) and 30 μl of internal standard (Table 4) were added, incubating by vortex at room temperature for 30 minutes and magnetically separated, the supernatant was discarded, 200 μl of 10% methanol/90% water was added to re-suspend the antibody magnetic beads, vortex was applied for 1 minute, the supernatant was discarded, the magnetic beads were washed twice with 200 μl of 10% methanol/90% water, 50 μl of 80% methanol/20% water was added to re-suspend the antibody magnetic beads, vortex was applied for 1 minute, the hormones on the antibody magnetic beads were eluted, magnetic separation was performed, 50 μl of the supernatant was transferred to a 96-well plate, and 50 μl of water was added, followed by uniform mixing and test on the Waters TQS mass spectrometer.

3. Optimization of the enrichment method: two enrichment reaction systems and washing systems of PBS and 10% methanol were compared respectively, enrichment was performed according to the above experimental methods, and the peak area of each hormone compound under the two conditions was tested.

### Test Results:

As shown in Table 6, the tests of 7 hormones including aldosterone, cortisol, testosterone, 11-deoxycortisol, androstenedione, corticosteroid, and 11-deoxycorticosterone found that compared with a single antibody-coupled magnetic bead, the peak area of each compound obtained by enrichment of the four antibody-coupled magnetic beads after mixing was larger; the peak area of the compounds obtained by the 10% methanol group was larger than that of the PBS group.

TABLE 6
Peak area of each hormone in enrichment reaction systems and antibody

| Type of | Enrichment | | | | | | | |
|---|---|---|---|---|---|---|---|---|
| antibody-coupled | reaction | | | | | | | |
| magnetic beads | system | Aldosterone | Cortisol | Testosterone | 11-deoxycortisol | Androstenedione | Cortin | 11-deoxycorticosterone |
| Cortisol | 10% | 20453.99 | 681982.81 | 267568.91 | 304872.50 | 248320.86 | 396689.91 | 919289.44 |
| Aldosterone | methanol | 145311.92 | 364919.09 | 266847.88 | 199970.77 | 245815.39 | 344649.47 | 896857.69 |
| Testosterone | | 17058.86 | 333941.22 | 1900920.00 | 181689.27 | 232164.25 | 319369.00 | 803196.31 |
| Progesterone | | 17930.73 | 333710.09 | 256170.69 | 189550.73 | 239844.09 | 322803.28 | 916500.94 |
| Mixture of 4 | | 156802.09 | 723380.13 | 2342727.50 | 801071.63 | 845156.06 | 827468.44 | 3170182.75 |
| antibodies | | | | | | | | |
| Cortisol | PBS | 7545.96 | 432575.81 | 180404.31 | 195862.11 | 127608.39 | 191395.14 | 55946.19 |
| Aldosterone | | 121955.14 | 92226.50 | 135283.30 | 94045.17 | 165512.77 | 115310.57 | 589176.00 |
| Testosterone | | 3798.76 | 54669.60 | 1715626.63 | 99455.63 | 176485.64 | 95048.06 | 631749.19 |
| Progesterone | | 4588.62 | 65679.71 | 190985.98 | 101755.56 | 169674.08 | 103458.52 | 659942.31 |
| Mixture of 4 | | 104069.56 | 442762.94 | 1804701.00 | 466088.47 | 546797.63 | 423962.72 | 2052265.88 |
| antibodies | | | | | | | | |

### Mixing Experiments

### EXAMPLE 4

### Test the Retention Time and Linear Range of Hormone Small Molecule Compounds

The experiment process was the same as that of Example 3

### Test Results:

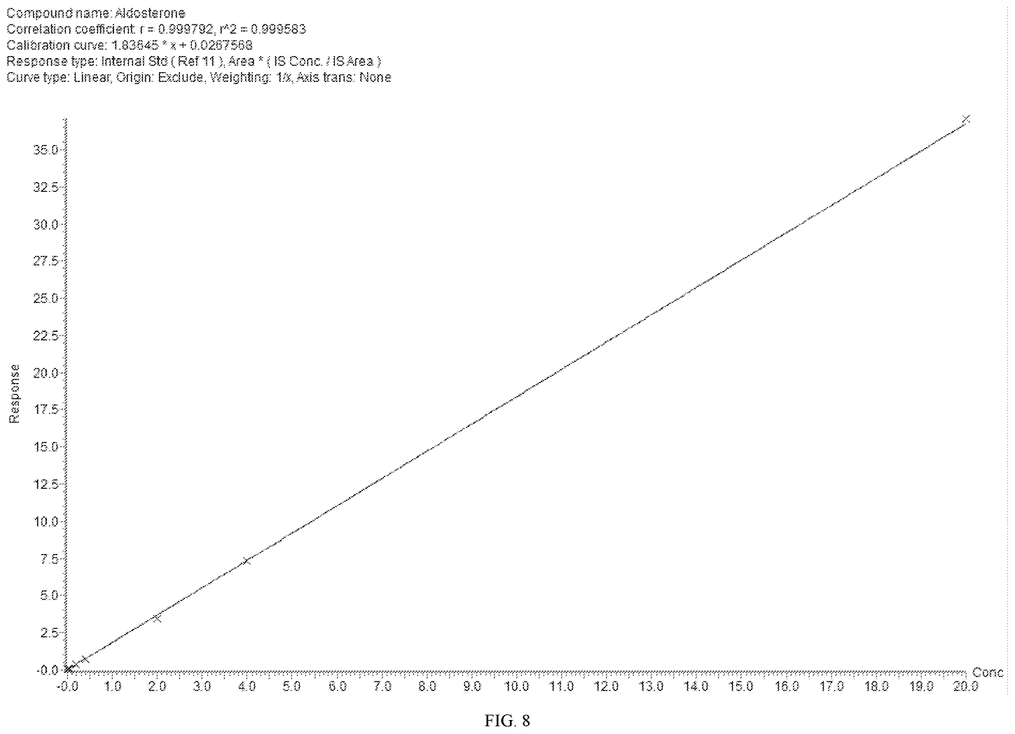

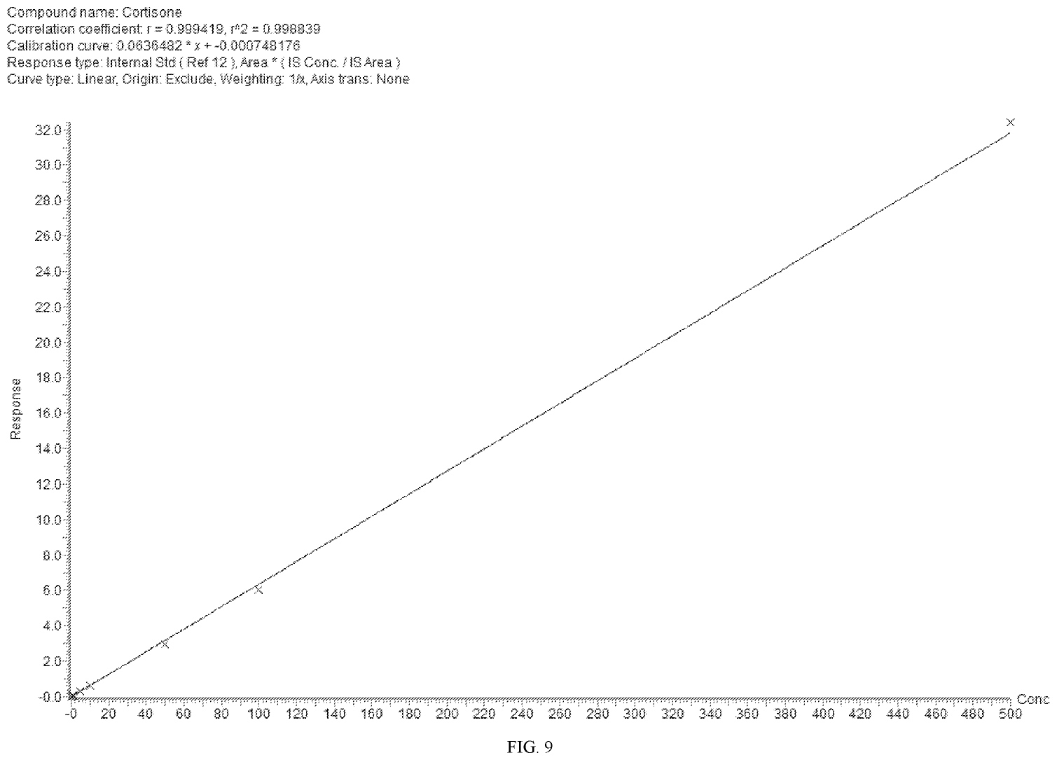

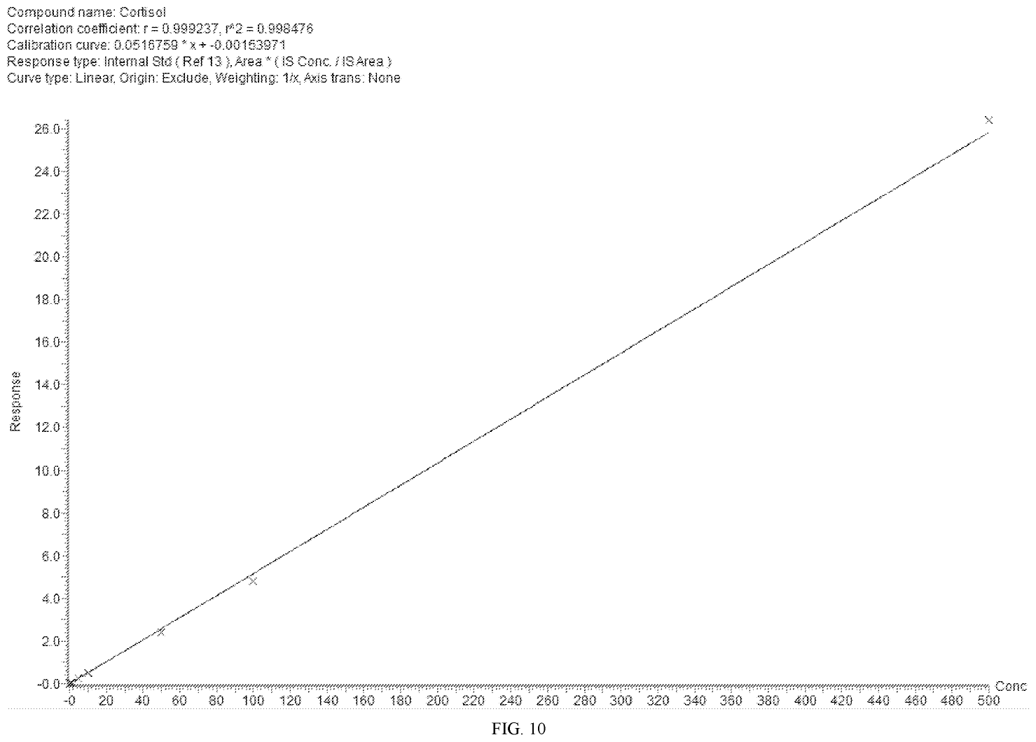

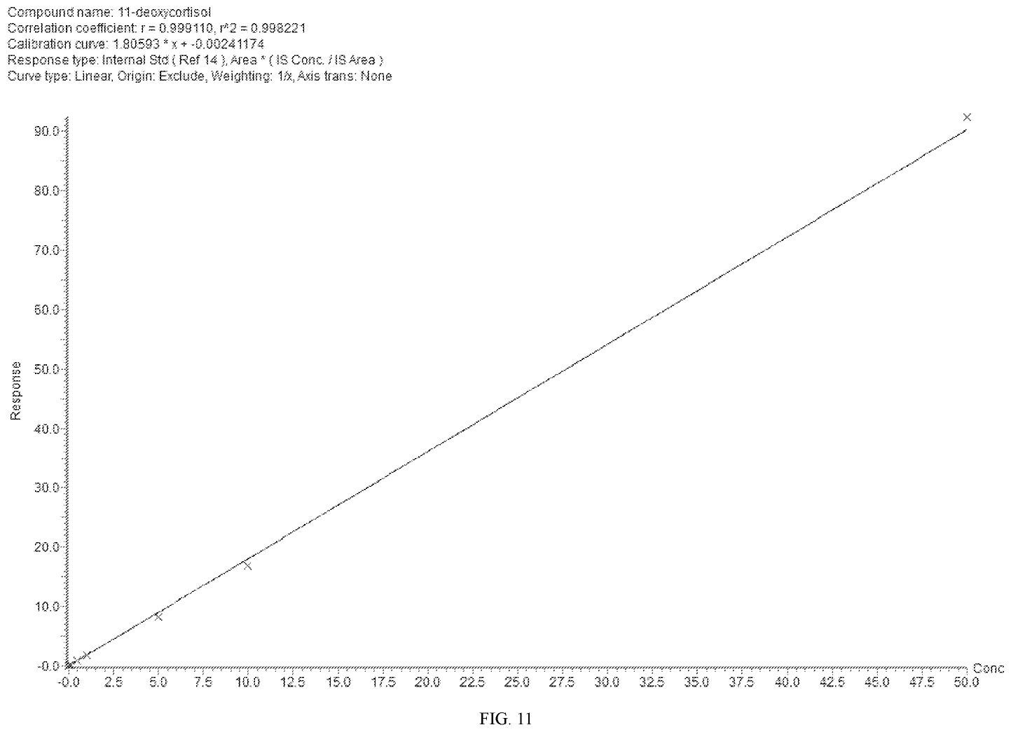

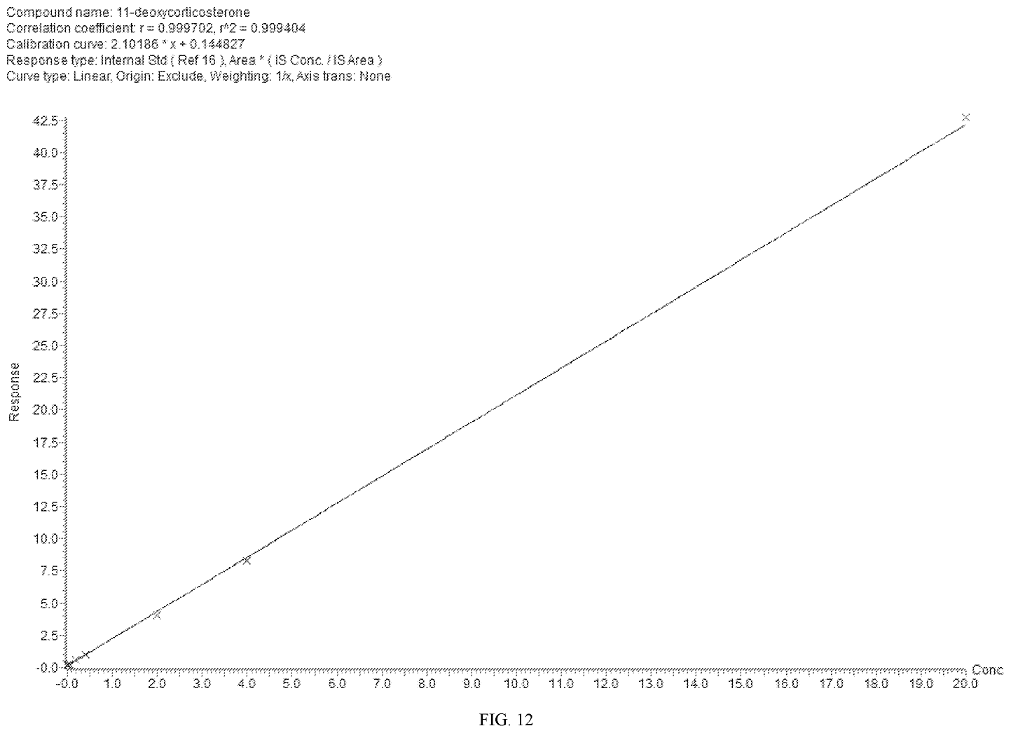

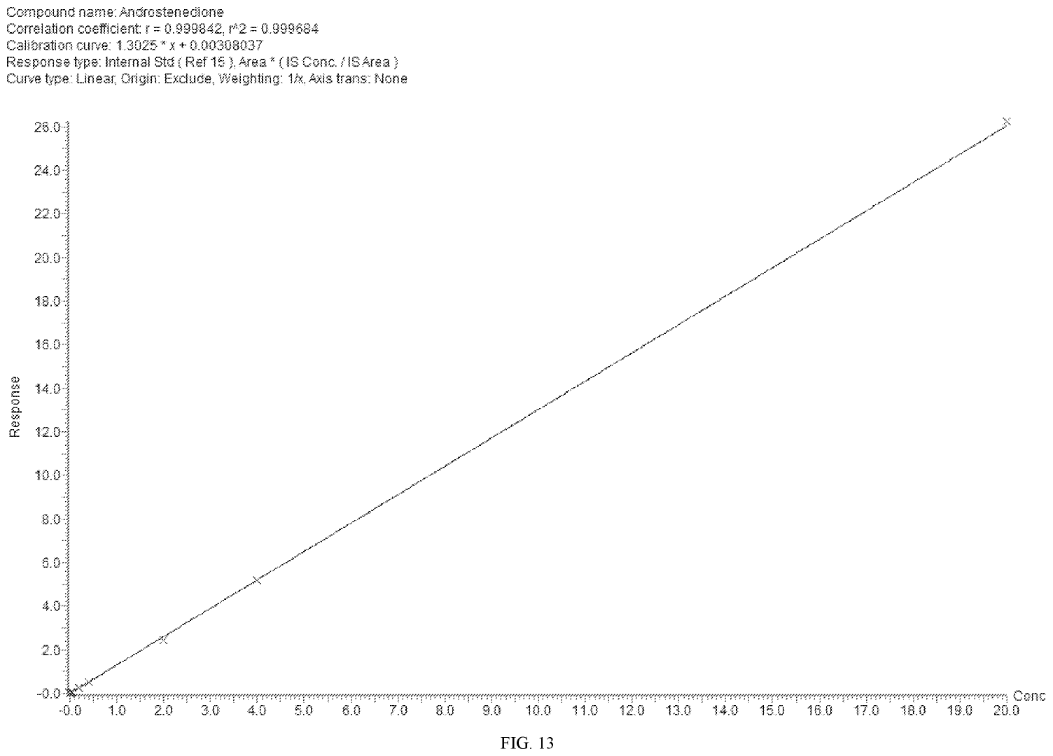

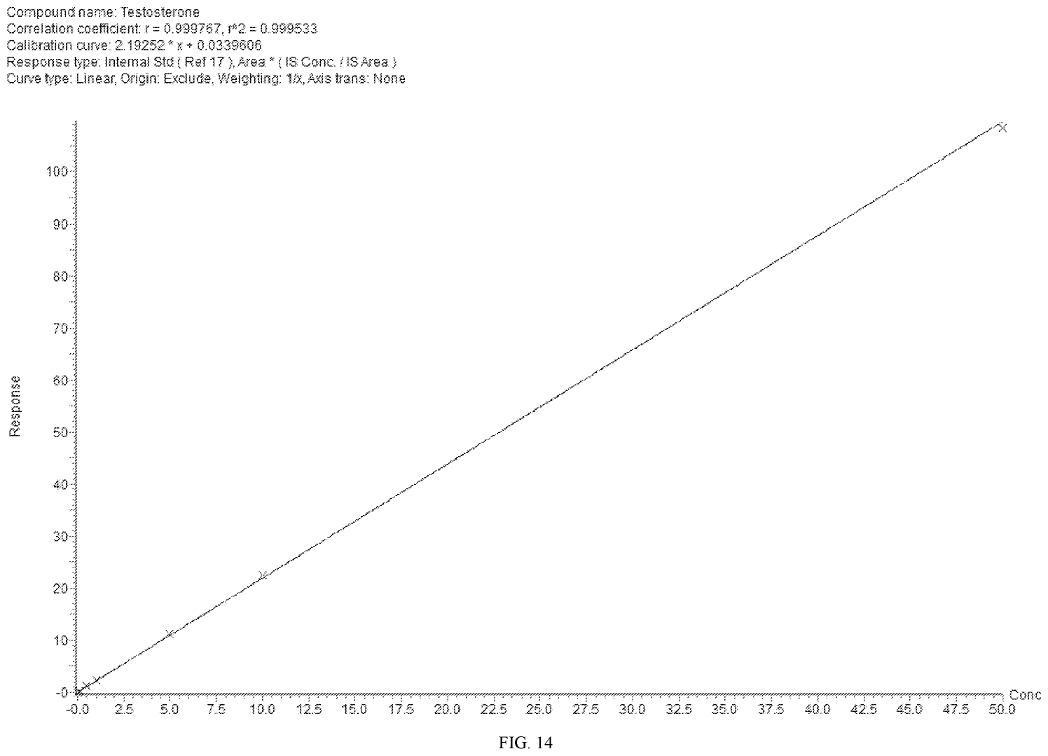

The retention time and linear range of the obtained hormone small molecule compounds were shown in Table 7, and the linear graphs were shown in FIGS. 8-14. From the experimental results, it can be seen that the correlation coefficient of each hormone compound is >0.99, indicating that the linear interval was acceptable and met the actual experimental requirements.

TABLE 7
Retention time and linear result of each hormone

| | | | | | Correlation |
|---|---|---|---|---|---|
| Serial | | Retention | Linear range | | coefficient |
| number | Target compound | time | (ng/ml) | Linear equation | (r2) |
| 1 | Aldosterone | 1.7 | 0.01-20.0 | y = 1.836x + 0.0268 | 0.9996 |
| 2 | Cortin | 2.02 | 0.25-500.0 | y = 0.064x − 0.001 | 0.9988 |
| 3 | Cortisol | 2.33 | 0.25-500.0 | y = 0.052x − 0.002 | 0.9985 |
| 4 | 11-deoxycortisol | 3.42 | 0.025-50.0 | y = 1.806x − 0.002 | 0.9982 |
| 5 | 11-deoxycorticosterone | 4.49 | 0.01-20.0 | y = 2.102x + 0.145 | 0.9994 |
| 6 | Androstenedione | 4.1 | 0.010-20.0 | y = 1.300x + 0.003 | 0.9997 |
| 7 | Testosterone | 4.63 | 0.025-50.0 | y = 2.192x − 0.034 | 0.9995 |

### EXAMPLE 5

### Standard Addition Recovery Experiment

### Test Process:

20 μl of four kinds of mixed 1 mg/ml antibody-coupled magnetic beads was taken, 200 μl of standard (Table 5) or serum sample and 30 μl of internal standard (Table 4) were added, incubating by shaking at room temperature for 30 minutes and magnetically separated, the supernatant was discarded, 200 μl of 10% methanol/90% water was added to re-suspend the antibody magnetic beads, vortex was applied for 1 minute, the supernatant was discarded, the magnetic beads were washed twice with 200 μl of 10% methanol/90% water, 50 μl of 80% methanol/20% water was added to re-suspend the antibody magnetic beads, vortex was applied for 1 minute, the hormones on the antibody magnetic beads were eluted, magnetic separation was performed, 50 μl of the supernatant was transferred to a 96-well plate, and 50 μl of water was added, followed by uniform mixing and test on the Waters TQS mass spectrometer. Each experiment was repeated 3 times, and the concentration value of each experimental group was calculated according to the standard curve. The recovery rate=(test sample concentration—basic sample concentration)/theoretical concentration×100%.

### Test Results:

The results of the standard addition recovery experiments were shown in Table 8 to Table 14. Judgment criteria of recovery experiments were: 80% <low-concentration average recovery rate <120% passed correctness verification, 85% <average recovery rate of medium and high concentrations <115% passed correctness verification, which showed that the experiment of this solution was feasible.

TABLE 8
Standard addition recovery results of aldosterone

| | Aldosterone | | | | | |
|---|---|---|---|---|---|---|
| | Low concentration (ng/ml) | | Medium concentration (ng/ml) | | High concentration (ng/ml) | |
| | Basic | Test | Basic | Test | Basic | Test |
| | sample | sample | sample | sample | sample | sample |
| First time | 0.000 | 0.032 | 0.038 | 9.862 | 0.038 | 14.816 |
| Second time | 0.000 | 0.034 | 0.038 | 9.847 | 0.038 | 14.539 |
| Third time | 0.000 | 0.035 | 0.038 | 9.726 | 0.038 | 14.785 |
| Average value | 0.00 | 0.03 | 0.04 | 9.81 | 0.04 | 14.71 |
| Test sample-basic sample | 0.034 | | 9.774 | | 14.675 | |
| Concentration of added standard sample | \ | 0.03 | \ | 10.00 | \ | 15.00 |

| | | | |
|---|---|---|---|
| Single concentration recovery rate % | 112.22 | 97.74 | 97.84 |

| | |
|---|---|
| Average recovery rate % | 102.60 |

TABLE 9
Standard addition recovery results of cortin

| | Cortin | | | | | |
|---|---|---|---|---|---|---|
| | Low concentration (ng/ml) | | Medium concentration (ng/ml) | | High concentration (ng/ml) | |
| | Basic | Test | Basic | Test | Basic | Test |
| | sample | sample | sample | sample | sample | sample |
| First time | 0.559 | 1.278 | 9.823 | 246.389 | 9.823 | 360.342 |
| Second time | 0.559 | 1.331 | 9.823 | 242.477 | 9.823 | 366.997 |
| Third time | 0.559 | 1.330 | 9.823 | 254.482 | 9.823 | 353.425 |
| Average value | 0.56 | 1.31 | 9.82 | 247.78 | 9.82 | 360.25 |
| Test sample-basic sample | 0.75 | | 237.96 | | 350.43 | |
| Concentration of added standard sample | \ | 0.75 | \ | 250.00 | \ | 375.00 |

| | | | |
|---|---|---|---|
| Single concentration recovery rate % | 100.53 | 95.18 | 93.45 |

| | |
|---|---|
| Average recovery rate % | 96.39 |

TABLE 10
Standard addition recovery results of cortisol

| | Cortisol | | | | | |
|---|---|---|---|---|---|---|
| | Low concentration (ng/ml) | | Medium concentration (ng/ml) | | High concentration (ng/ml) | |
| | Basic | Test | Basic | Test | Basic | Test |
| | sample | sample | sample | sample | sample | sample |
| First time | 2.847 | 3.370 | 55.967 | 272.941 | 55.967 | 403.544 |
| Second time | 2.847 | 3.430 | 55.967 | 277.950 | 55.967 | 396.791 |
| Third time | 2.847 | 3.505 | 55.967 | 280.609 | 55.967 | 386.460 |
| Mean | 2.85 | 3.44 | 55.97 | 277.17 | 55.97 | 395.60 |
| Test sample-basic sample | 0.59 | | 221.20 | | 339.63 | |
| Concentration of added standard sample | \ | 0.75 | \ | 250.00 | \ | 375.00 |

| | | | |
|---|---|---|---|
| Single concentration recovery rate % | 78.40 | 88.48 | 90.57 |

| | |
|---|---|
| Average recovery rate % | 85.82 |

TABLE 11
Standard addition recovery results of 11-deoxycortisol

| | 11-deoxycortisol | | | | | |
|---|---|---|---|---|---|---|
| | Low concentration (ng/ml) | | Medium concentration (ng/ml) | | High concentration (ng/ml) | |
| | Basic | Test | Basic | Test | Basic | Test |
| | sample | sample | sample | sample | sample | sample |
| First time | 0.013 | 0.099 | 0.234 | 23.037 | 0.234 | 32.997 |
| Second time | 0.013 | 0.100 | 0.234 | 24.453 | 0.234 | 32.789 |
| Third time | 0.013 | 0.098 | 0.234 | 22.867 | 0.234 | 34.051 |
| Mean | 0.01 | 0.10 | 0.23 | 23.45 | 0.23 | 33.28 |
| Test sample-basic sample | 0.09 | | 23.22 | | 33.05 | |
| Concentration of added standard sample | \ | 0.08 | \ | 25.00 | \ | 37.50 |

| | | | |
|---|---|---|---|
| Single concentration recovery rate % | 114.67 | 92.87 | 88.12 |

| | |
|---|---|
| Average recovery rate % | 98.55 |

TABLE 12
Standard addition recovery results of 11-deoxycorticosterone

| | 11-deoxycorticosterone | | | | | |
|---|---|---|---|---|---|---|
| | Low concentration (ng/ml) | | Medium concentration (ng/ml) | | High concentration (ng/ml) | |
| | Basic | Test | Basic | Test | Basic | Test |
| | sample | sample | sample | sample | sample | sample |
| First time | 0.011 | 0.044 | 0.160 | 10.085 | 0.160 | 14.587 |
| Second time | 0.011 | 0.043 | 0.160 | 9.769 | 0.160 | 14.831 |
| Third time | 0.011 | 0.047 | 0.160 | 9.915 | 0.160 | 14.366 |
| Mean | 0.01 | 0.04 | 0.16 | 9.92 | 0.16 | 14.59 |
| Test sample-basic sample | 0.034 | | 9.76 | | 14.43 | |
| Concentration of added standard sample | \ | 0.030 | \ | 10.00 | \ | 15.00 |

| | | | |
|---|---|---|---|
| Single concentration recovery rate % | 112.22 | 97.63 | 96.23 |

| | |
|---|---|
| Average recovery rate % | 102.03 |

TABLE 13
Standard addition recovery results of androstenedione

| | Androstenedione | | | | | |
|---|---|---|---|---|---|---|
| | Low concentration (ng/ml) | | Medium concentration (ng/ml) | | High concentration (ng/ml) | |
| | Basic | Test | Basic | Test | Basic | |
| | sample | sample | sample | sample | sample | Test sample |
| First time | 0.031 | 0.060 | 0.556 | 10.479 | 0.556 | 15.262 |
| Second time | 0.031 | 0.064 | 0.556 | 10.353 | 0.556 | 14.889 |
| Third time | 0.031 | 0.064 | 0.556 | 10.416 | 0.556 | 14.377 |
| Mean | 0.03 | 0.06 | 0.56 | 10.42 | 0.56 | 14.84 |
| Test sample-basic sample | 0.03 | | 9.86 | | 14.29 | |
| Concentration of added standard sample | \ | 0.03 | \ | 10.00 | \ | 15.00 |

| | | | |
|---|---|---|---|
| Single concentration recovery rate % | 105.56 | 98.60 | 95.24 |

| | |
|---|---|
| Average recovery rate % | 99.80 |

TABLE 14
Standard addition recovery results of testosterone

| | Testosterone | | | | | |
|---|---|---|---|---|---|---|
| | Low concentration (ng/ml) | | Medium concentration (ng/ml) | | High concentration (ng/ml) | |
| | Basic | Test | Basic | Test | Basic | Test |
| | sample | sample | sample | sample | sample | sample |
| First time | 0.024 | 0.110 | 0.618 | 26.006 | 0.618 | 38.409 |
| Second time | 0.024 | 0.113 | 0.618 | 26.063 | 0.618 | 37.759 |
| Third time | 0.024 | 0.113 | 0.618 | 26.086 | 0.618 | 36.222 |
| Mean | 0.02 | 0.11 | 0.62 | 26.05 | 0.62 | 37.46 |
| Test sample-basic sample | 0.088 | | 25.43 | | 36.85 | |
| Concentration of added standard sample | \ | 0.075 | \ | 25.00 | \ | 37.50 |

| | | | |
|---|---|---|---|
| Single concentration recovery rate % | 117.33 | 101.73 | 98.25 |

| | |
|---|---|
| Average recovery rate % | 105.77 |

### EXAMPLE 6

### 5-Day Precision Test

Test process: Same as Example 5, high and low concentration level samples, repeated 3 times a day, continuous measurement for five days.

### Test Results:

The obtained method precisions were shown in Table 15 to Table 19. It can be seen that the intra-batch and inter-batch precision CV values of this solution were less than 10%, which met the experimental requirements.

TABLE 15
5-day precision of aldosterone

| Parallel | Aldosterone | | | | | | | | | |
|---|---|---|---|---|---|---|---|---|---|---|
| sample | Low concentration (ng/mL) | | | | | High concentration (ng/ml) | | | | |
| number | Day 1 | Day 2 | Day 3 | Day 4 | Day 5 | Day 1 | Day 2 | Day 3 | Day 4 | Day 5 |
| Repeat 1 | 0.032 | 0.033 | 0.033 | 0.034 | 0.035 | 14.816 | 14.606 | 15.089 | 15.306 | 14.504 |
| Repeat 2 | 0.034 | 0.032 | 0.034 | 0.033 | 0.035 | 14.539 | 13.764 | 13.504 | 13.864 | 13.671 |
| Repeat 3 | 0.035 | 0.033 | 0.031 | 0.033 | 0.034 | 14.785 | 13.844 | 13.969 | 13.874 | 13.639 |
| Intra-batch average | 0.034 | 0.033 | 0.033 | 0.033 | 0.035 | 14.713 | 14.071 | 14.187 | 14.348 | 13.938 |
| Intra-batch standard deviation | 0.002 | 0.001 | 0.002 | 0.001 | 0.001 | 0.152 | 0.465 | 0.815 | 0.830 | 0.490 |
| Intra-batch precision CV (%) | 4.537 | 1.767 | 4.676 | 1.732 | 1.665 | 1.032 | 3.303 | 5.743 | 5.782 | 3.519 |

| | | |
|---|---|---|
| Inter-batch average | 0.033 | 14.252 |
| Inter-batch standard deviation | 0.001 | 0.582 |
| Inter-batch precision CV (%) | 3.543 | 4.081 |

TABLE 16
5-day precision of corticosteroid

| Parallel | Cortin | | | | | | | | | |
|---|---|---|---|---|---|---|---|---|---|---|
| sample | Low concentration (ng/mL) | | | | | High concentration (ng/ml) | | | | |
| number | Day 1 | Day 2 | Day 3 | Day 4 | Day 5 | Day 1 | Day 2 | Day 3 | Day 4 | Day 5 |
| Repeat 1 | 1.278 | 1.307 | 1.389 | 1.495 | 1.346 | 360.342 | 370.581 | 369.651 | 371.772 | 368.068 |
| Repeat 2 | 1.331 | 1.415 | 1.331 | 1.390 | 1.259 | 366.997 | 355.218 | 334.972 | 349.301 | 350.615 |
| Repeat 3 | 1.330 | 1.227 | 1.287 | 1.336 | 1.246 | 353.425 | 335.892 | 343.747 | 337.865 | 340.023 |
| Intra-batch average | 1.313 | 1.316 | 1.336 | 1.407 | 1.284 | 360.255 | 353.897 | 349.457 | 352.979 | 352.902 |
| Intra-batch standard deviation | 0.030 | 0.094 | 0.051 | 0.081 | 0.054 | 6.786 | 17.382 | 18.031 | 17.250 | 14.162 |
| Intra-batch precision CV (%) | 2.309 | 7.167 | 3.830 | 5.746 | 4.236 | 1.884 | 4.912 | 5.160 | 4.887 | 4.013 |

| | | |
|---|---|---|
| Inter-batch average | 1.331 | 353.898 |
| Inter-batch standard deviation | 0.071 | 13.440 |
| Inter-batch precision CV (%) | 5.297 | 3.798 |

TABLE 17
5-day precision of 11-deoxycorticosterone

| Parallel | 11-deoxycorticosterone | | | | | | | | | |
|---|---|---|---|---|---|---|---|---|---|---|
| sample | Low concentration (ng/mL) | | | | | High concentration (ng/ml) | | | | |
| number | Day 1 | Day 2 | Day 3 | Day 4 | Day 5 | Day 1 | Day 2 | Day 3 | Day 4 | Day 5 |
| Repeat 1 | 0.044 | 0.041 | 0.038 | 0.039 | 0.039 | 14.587 | 15.320 | 14.921 | 15.319 | 15.672 |
| Repeat 2 | 0.043 | 0.042 | 0.035 | 0.039 | 0.039 | 14.831 | 14.462 | 14.373 | 14.736 | 14.680 |
| Repeat 3 | 0.047 | 0.041 | 0.040 | 0.041 | 0.042 | 14.366 | 14.457 | 14.298 | 14.566 | 14.613 |
| Intra-batch average | 0.045 | 0.041 | 0.038 | 0.040 | 0.040 | 14.595 | 14.746 | 14.531 | 14.874 | 14.988 |
| Intra-batch standard deviation | 0.002 | 0.001 | 0.003 | 0.001 | 0.002 | 0.233 | 0.497 | 0.340 | 0.395 | 0.593 |
| Intra-batch precision CV (%) | 4.660 | 1.397 | 6.681 | 2.911 | 4.330 | 1.594 | 3.369 | 2.341 | 2.655 | 3.957 |

| | | |
|---|---|---|
| Inter-batch average | 0.041 | 14.747 |
| Inter-batch standard deviation | 0.003 | 0.404 |
| Inter-batch precision CV (%) | 6.934 | 2.737 |

TABLE 18
5-day precision of androstenedione

| Parallel | Androstenedione | | | | | | | | | |
|---|---|---|---|---|---|---|---|---|---|---|
| sample | Low concentration (ng/mL) | | | | | High concentration (ng/ml) | | | | |
| number | Day 1 | Day 2 | Day 3 | Day 4 | Day 5 | Day 1 | Day 2 | Day 3 | Day 4 | Day 5 |
| Repeat 1 | 0.060 | 0.064 | 0.066 | 0.062 | 0.062 | 15.262 | 15.829 | 15.629 | 15.815 | 15.723 |
| Repeat 2 | 0.064 | 0.065 | 0.065 | 0.063 | 0.062 | 14.889 | 15.087 | 15.194 | 15.093 | 15.296 |
| Repeat 3 | 0.064 | 0.060 | 0.061 | 0.061 | 0.058 | 14.377 | 15.100 | 14.969 | 15.359 | 15.459 |
| Intra-batch average | 0.063 | 0.063 | 0.064 | 0.062 | 0.061 | 14.843 | 15.339 | 15.264 | 15.422 | 15.493 |
| Intra-batch standard deviation | 0.002 | 0.003 | 0.003 | 0.001 | 0.002 | 0.444 | 0.425 | 0.336 | 0.365 | 0.215 |
| Intra-batch precision CV (%) | 3.685 | 4.200 | 4.134 | 1.613 | 3.807 | 2.994 | 2.769 | 2.198 | 2.368 | 1.391 |

| | | |
|---|---|---|
| Inter-batch average | 0.062 | 15.272 |
| Inter-batch standard deviation | 0.002 | 0.389 |
| Inter-batch precision CV (%) | 3.573 | 2.548 |

TABLE 19
5-day precision of testosterone

| Parallel | Testosterone | | | | | | | | | |
|---|---|---|---|---|---|---|---|---|---|---|
| sample | Low concentration (ng/mL) | | | | | High concentration (ng/ml) | | | | |
| number | Day 1 | Day 2 | Day 3 | Day 4 | Day 5 | Day 1 | Day 2 | Day 3 | Day 4 | Day 5 |
| Repeat 1 | 0.110 | 0.119 | 0.117 | 0.120 | 0.122 | 38.409 | 38.352 | 38.817 | 38.932 | 39.896 |
| Repeat 2 | 0.113 | 0.121 | 0.119 | 0.118 | 0.123 | 37.759 | 36.524 | 37.436 | 38.062 | 39.143 |
| Repeat 3 | 0.113 | 0.115 | 0.116 | 0.118 | 0.119 | 36.222 | 37.182 | 37.389 | 38.302 | 39.080 |
| Intra-batch average | 0.112 | 0.118 | 0.117 | 0.119 | 0.121 | 37.463 | 37.353 | 37.881 | 38.432 | 39.373 |
| Intra-batch standard deviation | 0.002 | 0.003 | 0.002 | 0.001 | 0.002 | 1.123 | 0.926 | 0.811 | 0.449 | 0.454 |
| Intra-batch precision CV (%) | 1.546 | 2.582 | 1.302 | 0.973 | 1.716 | 2.998 | 2.479 | 2.142 | 1.169 | 1.153 |

| | | |
|---|---|---|
| Inter-batch average | 0.118 | 38.100 |
| Inter-batch standard deviation | 0.004 | 1.021 |
| Inter-batch precision CV (%) | 3.065 | 2.680 |

The present invention is not limited to the above-mentioned best embodiments. Any person can derive other products in various forms under the enlightenment of the present invention. However, regardless of any change in shape or structure, all other technical solutions that are the same or similar to the technical solutions of the present application fall within the protection scope of the present invention.

1. A hormone mass spectrometric detection method based on antibody-coupled enrichment technology, comprising the following steps:
preparing antibody magnetic beads: coupling antibodies specific for hormone and magnetic beads, separating antibody beads and a supernatant by magnetic attraction, discarding the supernatant, adding a 50%-80% methanol solution and removing endogenous hormones carried on the antibodies by vortex, discarding the supernatant, then restoring the activity of the antibodies to obtain antibody magnetic beads that can enrich steroid hormone small molecule compounds, and adding test serum or a standard curve sample to obtain a first solution;
enriching test samples: adding an internal standard to the first solution and incubating at room temperature by vortex, and adding a 5-20% methanol solution for washing, to obtain antibody magnetic beads enriched with steroid hormone small molecule compounds;
eluting the test samples: placing the antibody magnetic beads enriched with steroid hormone small molecule compounds in an 80% methanol aqueous solution and re-suspending the same by vortex to obtain an eluent containing the steroid hormone small molecule compounds; and
testing the hormone small molecule compounds by mass spectrometry: placing the eluent under the set mass spectrometry and chromatography conditions for test;
wherein the antibody is included by cortisol antibody, testosterone antibody, aldosterone antibody and progesterone antibody;
wherein chromatographic conditions: models and specifications of chromatographic column and pre-column: Waters HSS T3 with a specification of 2.1×50 mm and 1.8 μm and HSS T3 with a specification of 2.1×5.0 mm and 1.8 μm; mobile phase A: 0.1% formic acid, 2 mM ammonium acetate aqueous solution; mobile phase B: 0.1% formic acid, 2 mM ammonium acetate methanol solution; gradient elution program: 0 minute: A: B=55: 45; 4.0 minutes: A:
B=40: 60; 6.5 minutes: A: B=25: 75; 7.5 minutes: A: B=10: 90; 7.6 minutes: A: B=55: 45; 8.0 minutes: A: B=55: 45; flow rate: 0.45 mL/min; column temperature: 45° C.; injection: 10 μL.

2. The hormone mass spectrometric detection method based on antibody-coupled enrichment technology according to claim 1, wherein the step of restoring the activity of the antibodies comprises: placing the antibody magnetic beads in organic solvent aqueous solutions with concentrations from high to low to restore the activity of the antibodies in a gradient manner.

3. The hormone mass spectrometric detection method based on antibody-coupled enrichment technology according to claim 1, wherein in the stage of enriching test samples, the antibody magnetic beads are used alone or in combination.

4. The hormone mass spectrometric detection method based on antibody-coupled enrichment technology according to claim 1, wherein in the step of enriching test samples, the methanol solution is 10% methanol solution.

5. The hormone mass spectrometric detection method based on antibody-coupled enrichment technology according to claim 1, wherein the antibodies are cortisol antibodies, testosterone antibodies, progesterone antibodies, and aldosterone antibodies.

6. The hormone mass spectrometric detection method based on antibody-coupled enrichment technology according to claim 1, wherein the magnetic beads are nano-scale carboxyl magnetic beads.

7. The hormone mass spectrometric detection method based on antibody-coupled enrichment technology according to claim 5, wherein the steroid hormone small molecule compounds are aldosterone, corticosteroid, cortisol, 11-deoxycortisol, 11-deoxycorticosterone, androstenedione and testosterone.

8. (canceled)

9. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 1 for hormone mass spectrometric detection.

10. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 2 for hormone mass spectrometric detection.

11. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 3 for hormone mass spectrometric detection.

12. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 4 for hormone mass spectrometric detection.

13. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 5 for hormone mass spectrometric detection.

14. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 6 for hormone mass spectrometric detection.

15. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 7 for hormone mass spectrometric detection.

16. A hormone mass spectrometric detection kit based on antibody-coupled enrichment technology, comprising:
magnetic beads, antibodies specific for hormone, a 50-80% methanol solution, a 5-20% methanol solution, organic solvent aqueous solutions with concentrations from high to low to restore the activity of the antibodies, and an 80% methanol solution, and using the hormone mass spectrometric detection method based on antibody-coupled enrichment technology according to claim 8 for hormone mass spectrometric detection.
